(19) United States
(12) Patent Application Publication
(10) Pub. No.: US 2012/0253218 A1
(43) Pub. Date: Oct. 4, 2012

(54) APPARATUS AND METHOD FOR EUCAPNIC VOLUNTARY HYPERVENTILATION TESTING

(76) Inventors: Richard R. Rosenthal, Great Falls, VA (US); Harvey J. Howe, Hartford, NC (US)

(21) Appl. No.: 13/343,694

(22) Filed: Jan. 4, 2012

Related U.S. Application Data
(60) Provisional application No. 61/429,756, filed on Jan. 4, 2011.

(51) Int. Cl. A61B 5/091; A61M 16/20

(52) U.S. Cl. 600/538

(57) ABSTRACT

A method and apparatus provide more efficient eucapnic voluntary hyperventilation (EVH) testing by using a low pressure demand valve that has low resistance during rapid breathing and by monitoring air flow to the subject from a pressurized tank using measurements of change in the tank's pressure. A second stage Scuba regulator is modified to provide a demand valve that has low resistance during rapid breathing.

This patent application claims priority from U.S. Provisional Application 61/429,756 of the same title filed on Jan. 4, 2011, which is incorporated herein by reference.

### BACKGROUND OF THE INVENTION

1. Field of the Invention

The present invention generally relates to testing for the condition of exercised induced bronchoconstriction (EIB), and in particular to improved test equipment and testing methods.

2. Background Description

Eucapnic voluntary hyperventilation was first established by Phillips et al. (“Phillips”) in 1983 for the purpose of exposing putative asthmatic subjects to a respiratory dose of CO₂ enriched dry air as a provocative challenge to demonstrate airways hyper reactivity. This procedure, known as an indirect type of challenge, is widely recognized as a surrogate for an exercise challenge which is done in order to demonstrate exercise induced bronchoconstriction in asthmatics who have characteristic episodes of bronchoconstriction after exercise.

The mechanism of exercise induced bronchoconstricton (EIB) is now recognized to be fundamentally due to the loss of mucosal water due to the drying effects of ventilated air passing over the respiratory mucosa (lining) of the bronchii. The attendant mucosal water loss causes a change in the osmotic milieu of the submucosal cells which thereby causes these cells to release histamine, prostaglandins and other chemical mediators which act on the bronchial smooth muscle and cause bronchospasm.

The voluntary hyperventilation of dry air simulates the stress of exercise induced hyperventilation and is used as a laboratory or clinical challenge to confirm or rule out the presence of exercise induced bronchospasm. Voluntary hyperventilation, however, will cause an inappropriate decrease in the dissolved. CO₂ content of the blood and a condition of hypocapnea will result. The consequential change in blood pH will often cause syncope or fainting of the individual hyperventilating. In order to prevent this, CO₂ may be added to the air voluntary hyperventilated in order to offset the unwanted CO₂ losses. It was determined by Phillips that a CO₂ level of 4.9% or 5% would be adequate to offset the CO₂ losses for all ventilatory rates between 40 and 105 liters per minute and that a gas mixture of 5% CO₂, 21% oxygen and 74% nitrogen could be safely used for eucapnic voluntary hyperventilation (EVH) challenges.

The Phillips apparatus for the conduct of EVH challenges consists of a tank of compressed breathing gas made up of 5% CO₂, 21% oxygen and 74% nitrogen. Compressed gas from the tank is vented either through a series of pressure reduction valves or demand valves to a rotameter which is used to regulate the flow of gas to a low compliance weather balloon which functions as a holding chamber for the gas at one atmosphere. A hose from the low compliance balloon goes to a low resistance two way mouthpiece from which the patient breathes at a target flow rate.

The target flow rate is generally determined from the FEV₁ (forced expiratory volume in one second) or volume of air the patient forcibly expels in one second having taken a baseline pulmonary function test prior to the challenge. Usually, the target flow rate per minute is 30 times the FEV₁, and the total volume of air to be given (known as the V_{E}) is the target flow rate for 6 minutes. For example if the FEV₁ is 3 liters, the target flow rate would be 90 liters per minute, and the total for 6 minutes would be a V_{E} of 540 liters.

In order to achieve the target flow rate, gas from the tank is valved into the balloon at the target flow rate V_{E} using the rotameter, and the patient must keep the balloon at a constant inflation in order to breathe at the prescribed flow rate. If the balloon is expanding, the patient is breathing or ventilating too slowly, if the balloon is collapsing, then the patient is ventilating too fast. This is very difficult to do accurately and hence the precision of the dosing of dry air is poor and there is no record of the actual V_{E}. Some operators in the literature have described a flow meter connected to the out-port of the two way valve in the patient's mouth. Moreover, there is no opportunity to monitor the patient during the test if it need be stopped prematurely should significant bronchospasm occur during the conduct of the challenge. After completion of the challenge, during which time the operator valved the target V_{E} into the balloon over a period of 6 minutes, a pulmonary function is repeated. This may be at 5 minute intervals for 20 minutes thereafter or some other sequence. The change, if any, in FEV₁ is taken as a measure of EIB. A 10% or greater decrease in FEV₁ is considered to be a positive test.

### SUMMARY OF THE INVENTION

It is therefore an object of the present invention to eliminate the meteorological balloon as a “plenum” for gas to be kept at ambient atmospheric pressure.

Another objective of the invention is to compute the target V_{E} based on the patient's baseline pulmonary function, and monitor and record the delivery of breathing gas in real time.

A further object of the invention to provide an accurate measure of the volume of gas delivered to the patient.

It is also an object of the invention to provide accurate feedback so that the patient is coached to maintain target airflow.

Another object of the invention is to allow for early discontinuation of the challenge according to operator determined criteria of diminished airflow through the bronchii.

An additional object of the invention is to stop the test when the target V_{E} has been reached and produce a report of all the test parameters and patient performance, and allow for an author generated interpretation of results.

The present invention consists of a means of reducing the tank pressure to ambient pressure (usually one atmosphere) without the use of an unwieldy meteorological balloon and enables precise patient feedback so as to achieve target ventilatory flow rates. It also enables the operator to monitor patient airflow during the conduct of the test and stop the test if flows drop because of concomitant bronchospasm during the test. Moreover it enables completion of the test when the total V_{E} has been administered to the patient. A record of the hyperventilation challenge including the total volume of gas ventilated, the minute by minute rate of ventilation, the time period needed to achieve the total target V_{E}, the pre and post test FEV₁, the percent change in FEV₁ and an interpretation of the test in terms of EIB absent, mild, moderate or severe.

The means by which this is accomplished may be described by reference to FIGS. 1-3 and consists of a (primary) regulator valve 130 fixed to the tank of compressed breathing gas 120 from which a demand valve of a SCUBA regulator type 140 may be attached by a low pressure hose 135 and used in a conventional way by the patient with an in-the-mouth fitting 145. The significance of adapting a breathing mechanism designed for underwater use is discussed in detail below. Also a second low pressure hose from the primary regulator 130 is fitted to an electronic pressure transducer 160, which in turn connects to a digital data acquisition unit 170. The data acquisition unit connects to a computer 180 which then records the static pressure in the tank before the beginning of the challenge and after, and sequential pressures during the conduct of the hyperventilation test. From the pressure changes, per unit time flow is computed and graphed. Flow determinations may be made continuously and the ventilator rate may also be monitored from the respiratory flow pattern. From this data a sloping line is displayed on the monitor screen and compared to the target slope (volume of gas ventilated/time) for the patient to see. From this graphic the patient is coached to attempt to match his ventilation rate to the target ventilation rate in terms of volume of gas ventilated per unit time.

A measured ventilatory rate significantly different from that of the first second (for example a 20% decrease in flow for a minute despite the same breathing frequency) may act as a warning threshold which signals the operator to stop the challenge because of probable intercurrent bronchospasm. The warning parameters can be user defined. When the target V_{E} has been achieved the test may be automatically terminated or alternatively the operator may elect to terminate the test when the 6 minute time period has elapsed. A static pressure in the tank 120 is determined at the end of the test.

A report is produced that contains the pretest and post test FEV₁ (entered by the operator). Alternatively, the data acquisition unit may be wired to a spirometer (not shown), an instrument used in parallel to the hyperventilometer, so that the FEV₁ data is entered into the EVH flowsheet and report automatically. The report will contain a record of the volume of air ventilated at multiple time points, for example each minute, and a graphic display of the V_{E} over time, and the total V_{E} ventilated. A record or any premature disruption of the test according to the operator set parameters will be included. Finally, an interpretation, (absent, mild, moderate or severe EIB or EVH induced bronchospasm) written according to established standards may be incorporated in the final report.

Applications for EVH challenge using the Hyperventilometer include 1) testing of patients for the presence of exercise induced asthma when the diagnosis is in doubt, 2) monitoring patient therapy for adequacy of antiasthma drugs, such as steroids, for attenuating the EVH challenge, 3) conduct of therapeutic drug trials for antiasthma drugs which block EIB (here exact duplication of the EVH challenge is critical as the patient response on and off investigational drug will be compared), 4) identifying athletes who have EIB so that they may be legitimately treated with antiasthma drugs before a sanctioned event (the International Olympic Committee considers EVH to be the gold standard by which elite athletes should be judged to have EIB), 5) qualifying or disqualifying would-be SCUBA divers and service academy candidates for training or enrollment on the basis of an asthma history, 6) qualifying for military duty in challenging ambient conditions, such as desert air, high performance aircraft, 7) assessment of disability claims, 8) evaluation of symptoms of asthma such as wheezing, shortness of breath or chest tightness in the face of normal pulmonary function tests, and other applications. The International Code of Diseases, edition 9 (ICD-9) recognizes EVH as a standard test for the evaluation of asthma.

In order for an athlete in a sanctioned event to be able to use an inhaled β₂ agonist prior to competing in the event, the International Olympic Committee Medical Commission requires notification in the form of a Therapeutic Use Exemption (TUE). The β₂ agonist is a standard antiasthma drug such as albuterol, usually delivered by inhalation using a metered dose inhaler. It is the policy of the Committee to require that athletes demonstrate the presence of asthma, exercise-induced bronchoconstriction (FIB), or airway hyper-reactivity (AHR). Eucapnic voluntary hyperventilation (EVH) is heavily favored by the Committee as the challenge test of choice used to demonstrate FIB. “EVH has been compared with exercise and other stimuli and is now well established for assessing elite athletes”. Fitch, Kenneth D., Sue-Chu, M., Anderson, S., Boulet, L. P., et., al., J Allergy Clin Immunol 2008; 122:254-60

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a schematic showing components of a hyper-ventilator system using a secondary tank.

FIG. 2 is a schematic showing components of a hyper-ventilator system using a secondary tank and a digital data acquisition unit.

FIG. 3 is a schematic showing components of a hyper-ventilator using a primary tank and a digital data acquisition unit.

FIG. 4A is a breathing resistance diagram for a second stage Scuba regulator.

FIG. 4B is a sample graph displaying a difference between actual patient ventilation 450 and a target ventilation 460 over a hyper-ventilation test period.

FIG. 5A is a mock-up of a computer screen for entering setup information for a patient hyper-ventilation test; FIG. 5B is a mock-up of test data and a post-test performance summary for a patient hyper-ventilation test.

### DETAILED DESCRIPTION OF A PREFERRED EMBODIMENT OF THE INVENTION

Survey data indicates that four out of five patients with asthma report one or more symptoms of exercise induced bronchospasm during or after sports, exercise, play or other physical activity. The rate of diagnosis is surprisingly low considering how widespread exercise induced bronchospasm is among asthmatics and the contribution or exercise-related symptoms to the burden of disease of asthma. Diagnosis of BIB using a convenient methodology such as the hyperventilometer (“Hyperventilometer”) described herein will help correct the apparent under-diagnosis reported in the survey data and facilitate more appropriate recognition and treatment of exercise induced asthma.

EVH is a particularly sensitive method of determining airways hyper-reactivity (AMR) insofar as it is very likely to be positive in most cases of FIB and it is very specific insofar as the rate of false positive determinations are expected to be low. Moreover, the EVH test with the Hyperventilometer lends itself to challenges of variable intensity, the selection of which will be a function of patient selection. The EVH challenge with the Hyperventilometer can be intensified by increasing the breathing rate or increasing the time of hyperventilation or both. In patients who are young, elderly, have lower baseline pulmonary functions, or are otherwise frail, lower respiratory rates or challenge times may be used. In elite athletes, a higher respiratory rate and or duration of challenge may be used and may be necessary to elicit evidence of EIB, should it exist. In all cases a record of challenge conditions and documentation of results will be provided.

The EVH challenge using the Hyperventilometer provides a means to follow the therapeutic status of a patient being treated for asthma as it is sensitive to conventional therapy. In this way medication can be adjusted according to the very objective findings of the EVH challenge rather than relying entirely on patient reported symptoms and as needed or otherwise prescribed medication use as an index of disease severity.

Because the conditions of challenge are very precise (ventilatory rate, total volume of air respired or V_{E} and time period) the identical EVH challenge may be re-administered on different occasions. This is highly desirable in a therapeutic drug trial under which circumstances the patient will be given the identical EVH challenge while taking the study drug or placebo.

Negative EVH challenges are used to qualify applicants with a questionable history of asthma for SCUBA instruction, recruitment in police, fire and military service, admission to military academies, and elimination of preexisting conditions for insurance coverage.

Examples of use of the invention for EVH testing include the following:

1. Using EVH as an EIB Surrogate.

Determination of exercise induced bronchospasm using EVH as a surrogate for an exercise challenge with treadmill, bicycle ergometer, field running, and similar exercise regimens. Physical fitness tests (PFTs) before and after the challenge are compared for signs of obstruction, measured by decreased forced exhalation flow rates. The diagnosis of exercise induced bronchospasm (EIB) would justify medical treatment before an event or even around-the-clock treatment for persistent symptoms.

2. Risk Assessment for SCUBA Diving.

The EVH challenge is essentially a SCUBA dive without the water. Dry air is hyperventilated which may cause bronchospasm in susceptible individuals. Bronchospasm during a dive may result in trapped air which upon expansion during ascent can cause rupture of the lung, arterial gas embolism, and related trauma.

In one example of this application, a fifteen year old female student who sought enrollment in a SCUBA diving class had an undocumented history of wheezing after a respiratory infection in the past, was on no medications presently and had normal pulmonary functions. Using the simplified method of the invention, she was given an EVH challenge with a target ventilation of 25 times her FEV₁ (62.5% of her estimated Maximum Voluntary Ventilation) for 6 minutes. She had a 24.8% decrease in her FEV₁ from baseline at 15 minutes after the challenge and required inhaled bronchodilator to restore her pulmonary function to baseline values. She was informed that she would be at risk of exercise induced bronchospasm triggered by the cold air of SCUBA exposure during actual dive conditions. This in turn would create the risk of arterial gas embolism, rupture of lung membranes, mediastinal emphysema or pneumothorax due to the expansion of trapped air upon ascent from depth and, accordingly, she was advised not to SCUBA dive.

3. Comparison with Mannitol Challenge.

Mannitol, a simple sugar, is very hygroscopic and when inspired as a dry powder, soaks up mucosal moisture and causes the same end results as breathing dry air would. Hence, inspiration of mannitol powder, too, is a surrogate for an exercise challenge, the natural consequence of which is to lose mucosal moisture because the tendency to humidify the incoming air. It is the water loss that causes histamine release from the tissues. The histamine in turn acts upon the bronchi to cause bronchospasm or narrowing of the bronchi. Mannitol (trade name Aridol) has been recently approved by the FDA and is available and marketed for the diagnosis of exercise induced bronchospasm.

However, a mannitol challenge does not have the specificity of EVH. Low specificity means there will be more false negatives. EVH may be compared to mannitol challenge in patients with exercise induced bronchospasm in order to better compare sensitivity and specificity of the two techniques. Low sensitivity means there will be more false positive results from a test.

4. Testing Vocal Cord Dysfunction.

Some patients have a paradoxical (inappropriate) adduction (coming together) of the vocal cords. There is a suggestion that this may be the consequence of inhaling dry air, similar to the bronchospasm secondary to exercise. An EVH challenge done at the same time as visualization of the vocal cords through a flexible fiberoptic pharangyscope inserted through the nose would help to elucidate this phenomenon in a way never possible before. Patients with vocal cord dysfunction would have EVH performed at the same time as nasopharyngoscopy with a video record to investigate this premise.

### Use of a SCUBA Regulator

A second stage SCUBA regulator is used as a means by which to reduce the tank pressure of up to 2000 psi to one atmosphere (ambient) so that it can be conveniently ventilated by the patient. The original EVH apparatus (Phillips, Y. Y., Jaeger, J., Laube, B. L., and Rosenthal, R. R., Eucapnic Voluntary Hyperventilation of Compressed Gas Mixture; Am Rev Respir Dis, 1985; 131:31-35) used a low resistance two way valve in order to vent the breathing gas from a tank of compressed gas to the patient by way of a compliant, meteorologic balloon that was used as a low pressure reservoir and then to provide for exhaust of the gas through the outport of the two way valve into the atmosphere or a gas volume measuring device such as a gas meter.

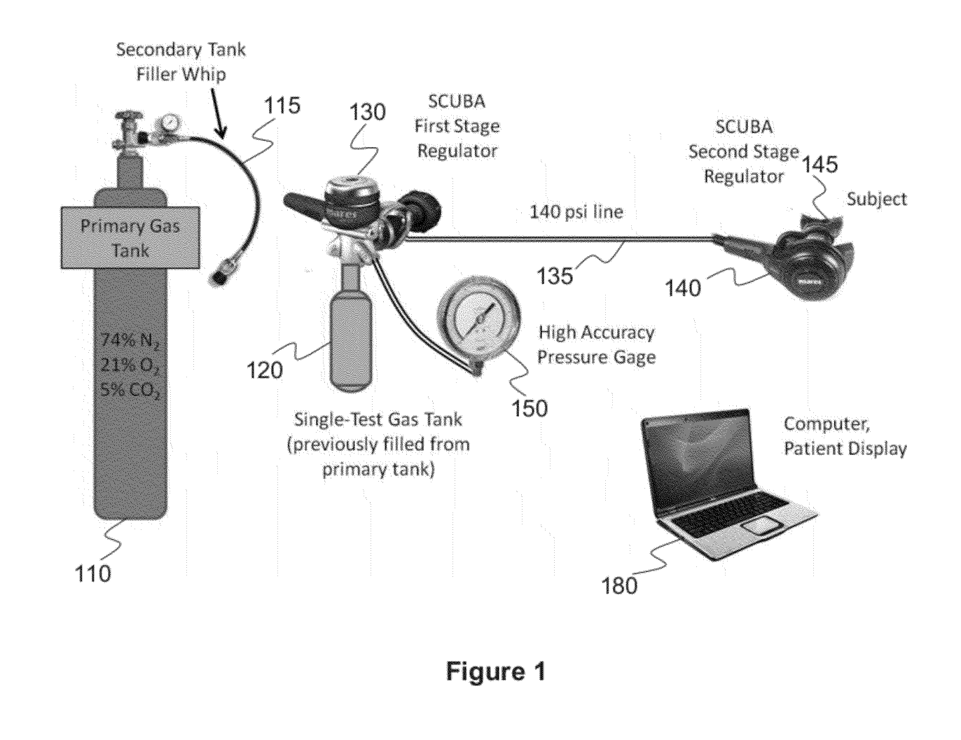

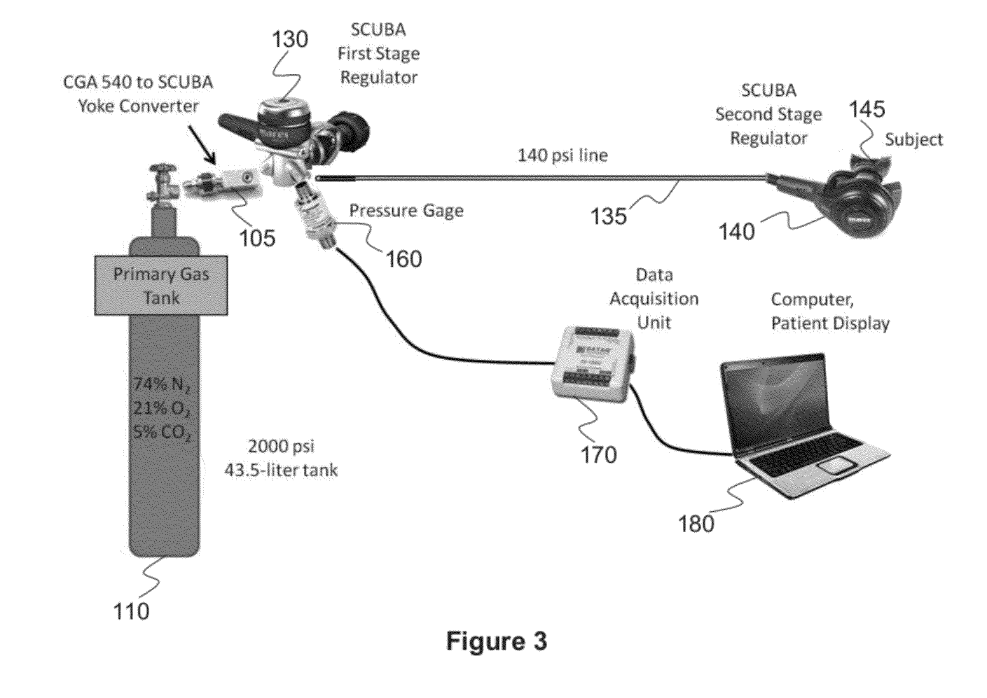

In the present invention, a very low resistance SCUBA regulator 140 is used instead of the meteorologic balloon and two way valve, and it is further modified such that the initial negative “cracking pressure” needed to open the valve to the air in the low pressure line 135 from the tank of compressed air (120 in FIGS. 1 and 2 and 110 in FIG. 3) is adjusted downward in order to further reduce the resistance of the SCUBA regulator. In this way, a previous caveat in the literature (Anderson, Argyros, G. J., Maghussen, H., Holzer, K., Br J Sports Med 2001; 35:344-347) about the unwelcome resistance of demand valves is overcome in such way as to eliminate the perceived disadvantage of demand valves and to leverage instead the advantage of such a valve, embodied in the modified SCUBA regulator. Such advantage being the elimination of an unwieldy low compliance meteorological balloon, the ability to easily administer target quantum of breathing gas to the patient, allow for patient feedback and coaching and for the regulation and recording of the EVH conditions of the challenge stimulus.

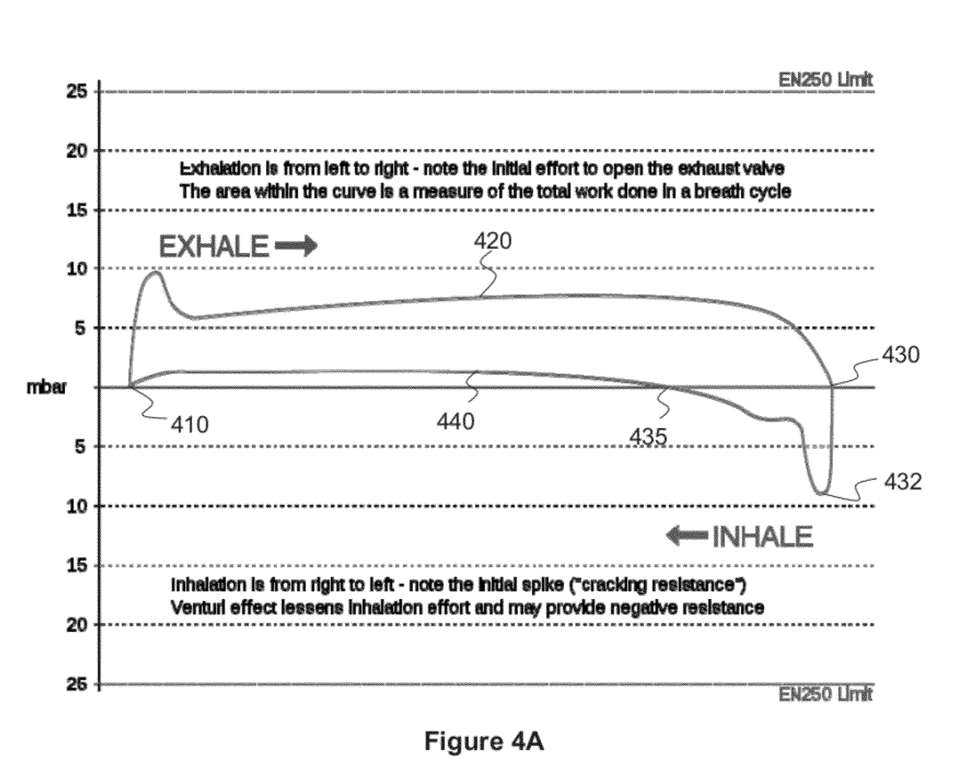

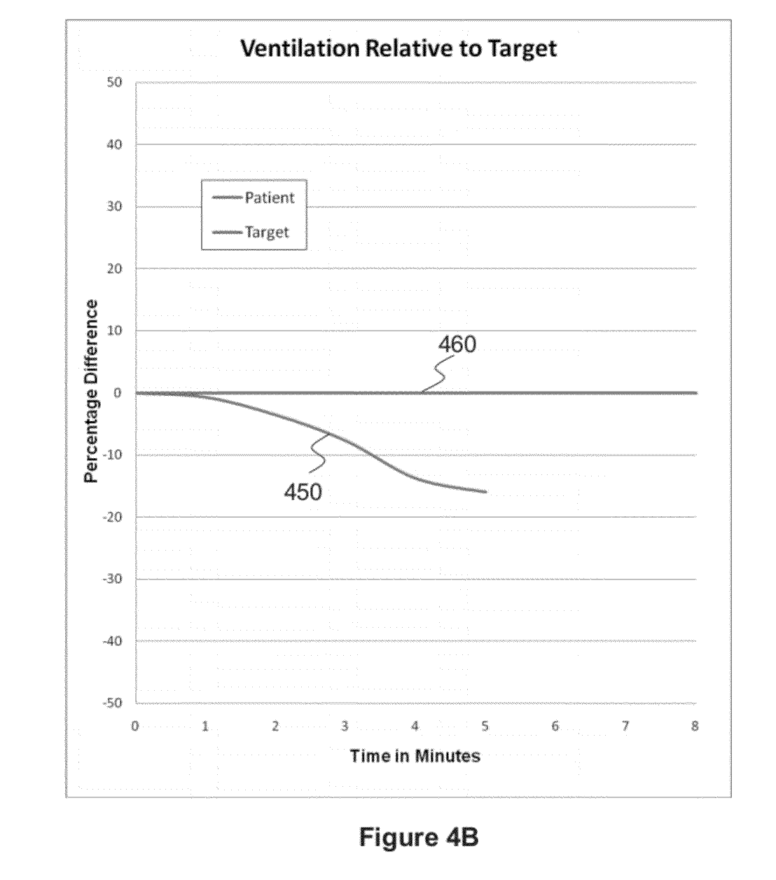

The advantages of these modifications of the SCUBA regulator may be understood by reference to the breathing resistance diagram shown in FIG. 4A, which applies to the second stage SCUBA regulator 140 which has mouthpiece 145 through which the subject breathes. Underneath the 0 mbar line is the scale 440 of negative inspiratory pressure (the pressure generated by the subject sucking in or inspiring during inhalation between 430 and 410) and superior to the 0 mbar line is the scale 420 of expiratory pressure needed to exhale (expiration from 410 to 430) through the mouthpiece 145 of the regulator 140. The caveat in the literature (Anderson) is that these valves offer high resistance during rapid breathing. One would equate the pressures required to ventilate (breath) through the SCUBA valve to such mentioned “resistance” as these pressures, both negative (inspiratory) and positive (expiratory) in millibars are required to overcome the resistances offered by the demand valve on inspiration and expiration.

At the far right, we note the spike downward 432 which means that the subject or patient must generate a negative inspiratory pressure of about 8 millibars in order to “crack open” the valve and initiate airflow through the valve from the low pressure (140 psi) line 135 coming into the valve 140 from the tank of air. The resistance overcome by this negative inspiratory pressure is called the “cracking resistance”. Once cracked open, air flows effortlessly to the subject (as the curve 440 moves to the left) and in fact a Venturi device built into the regulator 140 allows a little positive pressure ventilation to the subject (negative resistance) beginning at about 435 until at the far left of the graph (at 410) it is time to exhale.

In other words, there is a flow of air (between 435 and 410) from the tank through the valve to the patient which requires no effort or work on the part of the patient. At the far left of the graph (at 410) exhalation begins and requires some positive pressure to be produced by the patient on the order of 8 millibars, this time to open the exhaust valve and then push out the air until it is time to inhale again. Integrating the area “under the curve” is the “work of breathing” (which can be measured in joules). The point being that the purpose of generating these inspiratory and expiratory pressures is to overcome the resistance in the regulator caused by the intake valve and the exhaust valve.

To implement the invention the SCUBA regulator 140 is adjusted to minimize the “cracking pressure”. This adjustment makes the regulator inoperative for SCUBA diving, since it will be too easily triggered to provide airflow, but the positive airflow segment due to the Venturi assist during inspiration is a good thing for operation of the invention as it reduces the overall work of breathing. The work of exhalation is greatly minimized despite the need to overcome the resistance of the exhaust valve because at the end of the inhalation the subject has a full chest of air, having filled his lungs almost completely (not done in SCUBA diving which encourages quiet breathing). His lungs are full because he is hyperventilating taking repeated deep breaths in an attempt to do 80% of his predicted maximum voluntary ventilation—because this is a hyperventilation challenge. At the end of inhalation, the chest is full of air and elastic recoil of the chest wall and lungs will power the flow of air to open the exhaust valve and maintain airflow until it is time (at 430) for the inspiratory cycle to begin again.

So when we modify the SCUBA regulator 140 to reduce the cracking resistance, allow for the positive air flow due to the Venturi device (reducing the work of breathing which is the gray area under the curve—or between the curves), acknowledge that the normal elastic recoil of the lungs and chest wall will provide the kinetic energy to overcome the resistance of the exhaust valve, we conclude that this demand valve (modified second stage SCUBA regulator) does not in fact increase breathing resistance significantly over that of the conventional low resistance two way valve. Moreover, Anderson's statement that a demand valve offers more resistance with high ventilation rates is not applicable to the SCUBA regulator, as born out by the Breathing Resistance diagram shown in FIG. 4A, which neither predicts nor explains why the cracking resistance or the exhalation resistance should be a variable of breathing rate (rapid breathing) or flow rate sensitive.

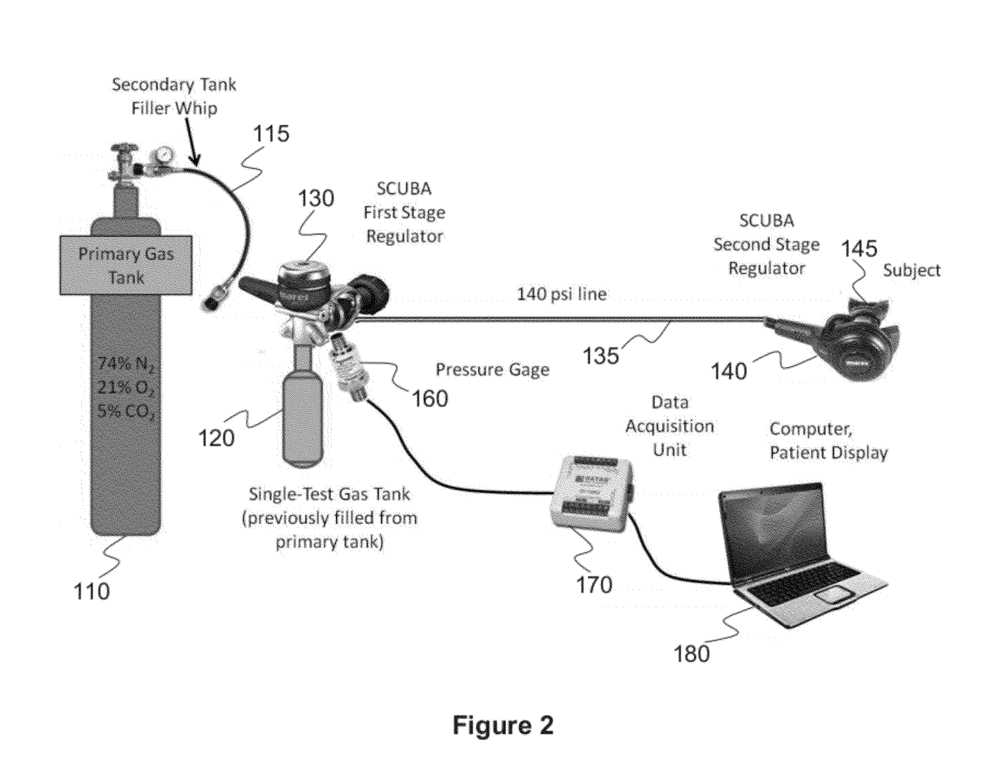

EVH has been established as a means to confirm exercise induced bronchoconstriction (EIB). An advanced, low cost system has been created to perform EVH evaluations. This Hyperventilator system may be implemented as depicted in FIGS. 1 and 2 below. Both implementations take advantage of the substantial cost benefits of integrating commercial of-the-shelf (COTS) components, including the use of a Scuba regulator whose properties have been adapted to the present invention. FIG. 1 shows the minimal, analog system used for evaluating the fundamental functionality of the design during the development process. Primary tank 110 contains a gas mixture of 74% nitrogen, 21% oxygen and 5% carbon dioxide. A filler whip 115 is used to fill a single-test gas tank 120 from the primary tank 110. High pressure gas from tank 120 flows through low pressure (140 psi) line 135 to the regulator 140.

FIG. 2 shows the digital version of the same system. The digital system has two advantages. First, data is recorded automatically at the appropriate times. Second, the pressure sensing and recording system may be sufficiently accurate to eliminate the need for a secondary, single-use tank (the need for which is discussed below), allowing direct use of primary tank 110 via a yoke converter 105 to connect to first stage regulator 130.

For EVH testing, the subject's ventilation rate is prescribed based on their pretest pulmonary performance (method for computing this value is well known). Ventilation rates over the entire test period need to be maintained near the prescribed level for the entire test period (usually six minutes). As seen in the depictions above, there is no flow rate or volume direct sensing device. Instead, volume flows are derived from measured tank pressures. The volume which is removed from the single-test gas tank 120 has a pressure relationship such that volume changes can be accurately estimated by pressure changes. There are several benefits of this technique over direct volume flow measurement. The first and most important benefit is cost. There are many types of sensors for the measurement of flow rates or integrated volumes but all are more costly than a pressure transducer.

Numerous types were researched including those that measure rate based on differential pressures measured after flow is forced to negotiate a known geometry. These include venturi tubes, pitot tubes and orifice plates. Other types have mechanical systems that are excited by the flow such as turbines, paddle wheels and positive displacement pistons. There are also flow cooling thermal devices like hot wire and ceramic anemometers. There are exotics like ultrasound, Coriolis, magnetic, and vortex shedding rate meters. Each of these types is more costly than a pressure transducer. (An exception is mass flow sensors for automobiles, which are very low cost because of the enormous numbers produced and competition. These units are far too large for conversion to this application.)

A second benefit for using a pressure sensor is simplicity of installation. It can be placed directly on the SCUBA first stage regulator at one of the high pressure ports. Volume flow measurement would require a sensor in the flow stream. There are three possible locations for such a flow sensor; they are between the tank and the SCUBA first stage regulator, between the first and second stage SCUBA regulators, and downstream of the patient. Between the regulators is not a good choice because there it may interfere with the dynamics of these regulators working together. Likewise, downstream of the patient is not desirable because of the complexity of the attachment, the additional restriction on patient movement, and the effects that any flow resistance of the sensor would have. Between the tank and the first stage regulator would work, but this is an area of high pressure. Any device and attaching hoses now must be safety certified which can drive up costs.

The only drawback for the pressure sensor is that it must be capable of measuring pressures accurately because of the relatively small change in pressures. For example, for a full tank, only 10% of the tank volume is used for a typical subject. If the limit of the error for flow measurement, is elected to be, say 5%, then the pressure would have to measured with 0.5% accuracy. This may be possible with even low cost pressure transducers, because the actual measurement would be a relatively small differential. Therefore, this accuracy is driven, by the sensor linearity more than absolute accuracy. The implementation shown in FIG. 3 can be used with a suitably linear sensor. It should be noted that further automation can be achieved by conducting the pulmonary function test (for FEV₁), which is done before and after the challenge, using a spirometer or other measuring device whose output is fed into the computer.

Alternatively, if the tank volume can be reduced to a single usage, nearly all the gas in the tank will, be used for a single test. This will greatly increase the accuracy of the prediction of the volume flow based on measured pressures. This is the baseline concept as shown in FIGS. 1 and 2.

The system shown in FIG. 1 is designed to deliver a prescribed dry, CO₂-rich, breathable gas mixture that is common to EVH testing. This mixture is available in large tanks delivered by bottled gas companies. Gas is transferred from this “primary” tank 110 to a smaller “secondary” tank 120 that is used for delivery to the subject. The secondary tank 120 is used for two purposes. First, it is small enough to use in a typical examination room and is easily transported. Secondly, it allows proportionally larger changes in pressure for the extraction of any given gas volume. This permits the use of a relatively low-cost pressure gage to accurately monitor gas volume in real time. It is possible, of course, to use a flow meter, such as a pneumotachograph, or other mechanical or electronic flow meter, to obtain a calculation of V_{E}.

For the implementation shown in FIG. 1, this pressure gage is a high accuracy analog gage 150. It is connected via a long high pressure hose to a high pressure port on the SCUBA first stage regulator 130 that is mounted on the tank head. In order to achieve the most accurate assessment of the vented gas volume (as inferred from the pressure gage readings) and to conserve gas, it is necessary to fill the secondary tank only with the amount of gas needed for the specific EHV evaluation being conducted. The volume of gas that is needed is based on the ventilation rate for a test period of six minutes. The fill pressures necessary to produce the required volumes have been calculated and are presented in the table below. Of course, the values are only valid for the 11.1 liter capacity secondary tank used in the initial implementation.

TABLE 1
Secondary Tank Target Fill Pressures

| Vent. Rate | Fill Pressure |
|---|---|
| l/m | psi |
| 20 | 299 |
| 30 | 378 |
| 40 | 458 |
| 50 | 537 |
| 60 | 617 |
| 70 | 696 |
| 80 | 776 |
| 90 | 855 |
| 100 | 935 |
| 110 | 1,014 |

The system should be set up as shown schematically in FIG. 1. The computer is shown in the setup because it is integral to data reduction and display. It is not connected to the data collection system as it is for the sully digital implementations shown in FIGS. 2 and 3. For the FIG. 1 implementation, pressure readings are made manually from the analog pressure gage 150 and data is entered on the computes 180.

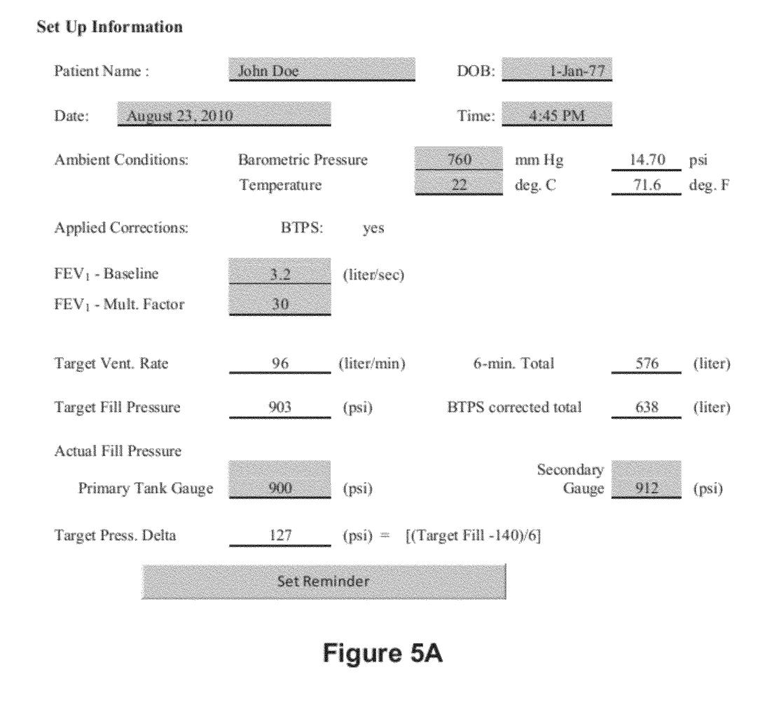

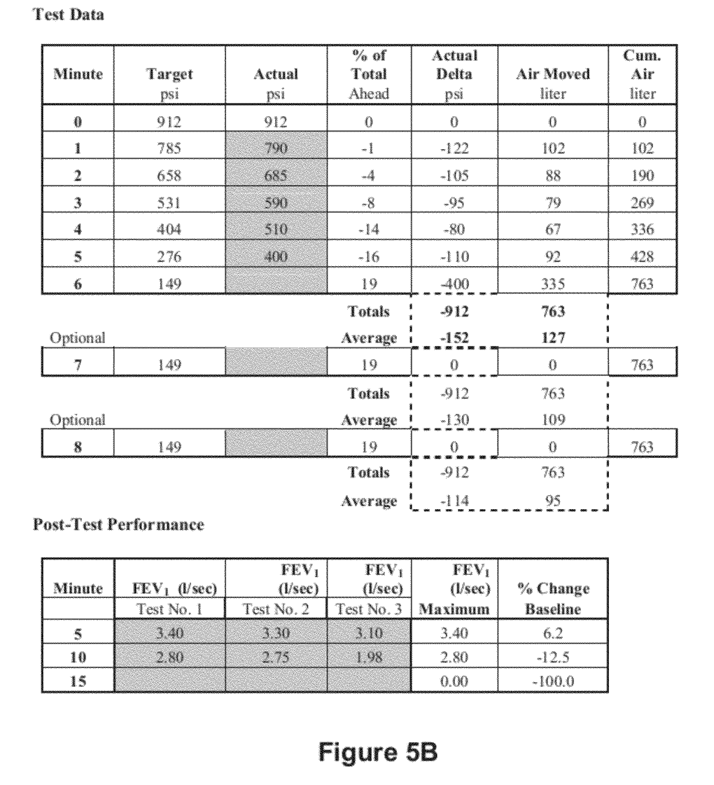

The prescribed ventilation rate will result in a corresponding pressure at the end of each minute of the test. The table in FIG. 5B shows this pressure for each of the six minutes of the test for all prescribed flow rates from 20 to 110 l/m. For visualization these pressures are also plotted versus time in the chart below. There pressures are a direct indication of the total quantity of gas ventilated up to any given point in time. At the end of each minute, the clinician will read the pressure from the secondary tank pressure gage and compare it to the scheduled pressure. They will then direct the subject accordingly. The success of this corrective action will be known at the end of the next minute test period when actual pressure is again compared to the scheduled pressure.

For the minimal system used during the system evaluation period, the secondary tank initial pressures prescribed in Table 1 above will be difficult to set accurately. This is because of the low resolution of the cage used during the fill process. For this circumstance, it may be easier for the clinician to attempt to achieve the designated pressure delta from one minute to the next. As seen in the data shown in FIG. 5B, this pressure is a single value for each of the six minutes of the evaluation event.

It is possible to apply the delta below using the pressure gage of the secondary tank because it has much more resolution than the fill gage and can easily be read to five psi accuracy. It is unlikely that the secondary tank will, be filled to the target pressure of Table 1 with poor precision. But once the secondary tank is prepared for testing, the high-accuracy pressure gage on it will provide sufficient resolution for metering the gas to the subject. A simple formula can be applied to calculate a delta for whatever fill pressure is measured on the secondary tank. This formula is:

Pressure Delta=(Fill Pressure−140)/6

This is the pressure delta that should be depleted from the tank at the end of each of the six minutes of the test. This will leave the tank with the minimum pressure for proper operation of the SCUBA secondary regulator. Calculating the target pressure for the end of the first minute for example, the pressure delta calculated above would be subtracted from the fill pressure. For the end of the second minute, the delta would be subtracted from the first minute value, and so on. These are the target pressures for the subject to attempt to achieve. It is up to the clinician to constantly provide the corrective feedback to bring the subject to these values.

While the invention has been described in terms of preferred embodiments, those skilled in the art will recognize that the invention can be practiced with modification within the spirit and scope of the appended claims.

Having thus described our invention, what we claim as new and desire to secure by Letters Patent is as follows:

1. A method for eucapnic voluntary hyperventilation (EVH) testing, comprising:
performing a baseline pulmonary function test on a subject to determine a baseline value of forced expiratory volume (FEV₁);
performing a eucapnic voluntary hyperventilation (EVH) challenge on the subject, further comprising:
providing a known amount of carbon dioxide enriched air at atmospheric pressure to the subject at a target flow rate, the known amount and the target flow rate being calculated from the baseline value, the known amount being provided from a pressurized container and the target flow rate being correlated to a change in pressure of the pressurized container;
using a low pressure demand valve for the subject to inhale and exhale the known amount of carbon dioxide enriched air at atmospheric pressure to meet the target flow rate, the demand valve having a low resistance during rapid breathing;
performing one or more follow-up pulmonary function tests on the subject in a specified sequence to determine a comparative value of the forced expiratory volume (FEV₁);
comparing the comparative value to the baseline value to determine a condition of the subject in response to the EVH challenge.

2. The method of claim 1, wherein the low pressure demand valve is a second stage Scuba regulator modified to reduce a “cracking pressure” to a low level unusable for Scuba diving.

3. The method of claim 1, wherein the pressurized container contains the known amount and the change in pressure is measured by a pressure gauge at one minute intervals during the EVH challenge.

4. The method of claim 1, wherein the pressurized container is a primary tank and the change in pressure is measured by a pressure gauge that is linear.

5. The method of claim 3, wherein the measured pressure is in digital form and is stored in a data acquisition unit.

6. The method of claim 5, wherein the pulmonary function tests are monitored using a spirometer and the monitored results are fed into the data acquisition unit for automatic determination of FEV₁ values.

7. The method of claim 1, wherein the flow rate is determined at regular intervals from changes in pressure.

8. The method of claim 7, wherein a drop in flow rate is used to determine a likely condition of bronchoconstriction in the subject and terminate the EVH challenge.

9. The method of claim 1, further comprising using the results of the comparing step to monitor adequacy of antiasthma drugs for attenuating the EVH challenge.

10. The method of claim 1, further comprising using the results of the comparing step to qualify or disqualify would-be Scuba divers.

11. A system for eucapnic voluntary hyperventilation (EVH) testing, comprising:
means for performing a baseline pulmonary function test on a subject to determine a baseline value of forced expiratory volume (FEV₁);
means for performing a eucapnic voluntary hyperventilation (EVH) challenge on the subject, further comprising:
means for providing a known amount of carbon dioxide enriched air at atmospheric pressure to the subject at a target flow rate, the known amount and the target flow rate being calculated from the baseline value, the known amount being provided from a pressurized container and the target flow rate being correlated to a change in pressure of the pressurized container;
a low pressure demand valve for the subject to inhale and exhale the known amount of carbon dioxide enriched air at atmospheric pressure to meet the target flow rate, the demand valve having a to resistance during rapid breathing;
means for performing one or more follow-up pulmonary function tests on the subject in a specified sequence to determine a comparative value of the forced expiratory volume (FEV₁); and
means for comparing the comparative value to the baseline value to determine a condition of the subject in response to the EVH challenge.

12. The system of claim 11, wherein the low pressure demand valve is a second stage Scuba regulator modified to reduce a “cracking pressure” to a low level unusable for Scuba diving.

13. The system of claim 11, wherein the pressurized container contains the known amount and the change in pressure is measured by a pressure gauge at one minute intervals during the EVH challenge.

14. The system of claim 11, wherein the pressurized container is a primary tank and the change in pressure is measured by a pressure gauge that is linear.

15. The system of claim 13, wherein the measured pressure is in digital form and is stored in a data acquisition unit.

16. The system of claim 15, wherein the pulmonary function tests are monitored using a spirometer and the monitored results are fed into the data acquisition unit for automatic determination of FEV₁ values.

17. The system of claim 11, wherein the flow rate is determined at regular intervals from changes in pressure.

18. The system of claim 17, wherein a drop in flow rate is used to determine a likely condition of bronchoconstriction in the subject and terminate the EVH challenge.

19. A method for performing a eucapnic voluntary hyperventilation (EVH) challenge on a subject, comprising:
providing a known amount of carbon dioxide enriched air at atmospheric pressure to the subject at a target flow rate, the known amount and the target flow rate being calculated from a forced expiratory volume (FEV₁) determined in a pulmonary function test of the subject, the known amount being provided from a pressurized container and the target flow rate being correlated to a change in pressure of the pressurized container;
using a low pressure demand valve for the subject to inhale and exhale the known amount of carbon dioxide enriched air at atmospheric pressure to meet the target flow rate, the demand valve having a low resistance during rapid breathing.

20. The method of claim 19, wherein the low pressure demand valve is a second stage Scuba regulator modified to reduce a “cracking pressure” to a low level unusable for Scuba diving.
